(19) United States
(12) Patent Application Publication
(10) Pub. No.: US 2019/0015131 A1
(43) Pub. Date: Jan. 17, 2019

(54) BONE ACCESS DEVICE HOLDER AND METHODS OF USE

(71) Applicant: Merit Medical Systems, Inc., South Jordan, UT (US)

(72) Inventors: Michael J. Hallisey, Wethersfield, CT (US); Jim Mottola, West Jordan, UT (US); Kenneth Sykes, Bluffdale, UT (US)

(21) Appl. No.: 16/035,022

(22) Filed: Jul. 13, 2018

Related U.S. Application Data
(60) Provisional application No. 62/532,717, filed on Jul. 14, 2017.

(51) Int. Cl. A61B 17/34; A61B 10/02; A61B 17/88

(52) U.S. Cl. CPC A61B 17/3423; A61B 10/025; A61B 17/7094; A61B 17/3472; A61B 17/8819; A61B 17/885

(57) ABSTRACT

Devices used to hold a tissue access device, such as a bone cannula, at an access site are disclosed. The devices may be configured to hold the access device in a stable orientation while keeping the hand of a medical practitioner outside of an x-ray radiation field. The devices further include a patient contact member to facilitate orientation of the tissue access device.

### RELATED APPLICATIONS

This application claims priority to U.S. Provisional Application No. 62/532,717, filed on Jul. 14, 2017 and titled “Bone Access Device Holder and Methods of Use” which is hereby incorporated by reference in its entirety.

### TECHNICAL FIELD

This present disclosure relates to medical instruments and systems for providing an access in vertebral bone to deliver bone cement to treat a vertebral compression fracture. However, the features relating to the methods and devices described herein can be applied in any tissue where access to the tissue is desired for ablation, biopsy or other medical procedure and x-ray radiation is utilized for imaging of the tissue.

### BRIEF DESCRIPTION OF THE DRAWINGS

The embodiments disclosed herein will become more fully apparent from the following description and appended claims, taken in conjunction with the accompanying drawings. The drawings depict only typical embodiments, which embodiments will be described with additional specificity and detail in connection with the drawings in which:

FIG. 1 is a perspective view of an access device holder.

FIG. 2 is a side view of the access device holder.

FIG. 3 is a partially cutaway bottom view of a holding device of the access device holder.

FIG. 4A is a partially cutaway bottom view of the holding device of the access device holder having cross-hairs targeting elements.

FIG. 4B is a partially cutaway bottom view of the holding device of the access device holder having concentric circles targeting elements.

FIG. 4C is a partially cutaway bottom view of the holding device of the access device holder having a sphere targeting element.

FIG. 5 is a side view of the access device holder having a telescoping shaft.

FIG. 6 is a side view of the access device holder with a surgical marking pen coupled to the holding device.

FIG. 6A is an illustration of a stamped skin marking image of a full disc.

FIG. 6B is an illustration of a traced skin marking image of the outline of the disc.

FIG. 6C is an illustration of a skin marking image of a dot.

FIG. 7A is a side view of the access device holder and an access device prior to insertion of the access device.

FIG. 7B is a side view of the access device holder and the access device subsequent to insertion of the access device into a vertebral bone.

FIG. 7C is a side view of the access device holder and the access device with a bone cement delivery system coupled to the access device.

FIG. 8 is a perspective view of an embodiment of the access device holder having a “U” shaped patient contact member.

FIG. 9 is a side view of another embodiment of the access device holder having an actuatable holding device.

FIG. 10A is a top view of a distal portion of the embodiment of FIG. 9 disposed in a secure configuration and engaged with an access device shaft having a relatively small diameter.

FIG. 10B is a top view of a distal portion of the embodiment of FIG. 9 disposed in a secure configuration and engaged with an access device shaft having a relatively larger diameter.

FIG. 10C is a top view of a distal portion of the embodiment of FIG. 9 disposed in a release configuration.

### DETAILED DESCRIPTION

Access to vertebral bone and other tissues is a common medical procedure performed to inject bone cement to treat vertebral fractures or for other procedures such as ablation or tissue biopsy. The access procedure often requires the utilization of x-ray radiation for imaging of the vertebral bone or tissues to accurately position an access device such as a cannula and trocar or biopsy needle. The hand of a medical practitioner is often in the radiation field. Low dose radiation is known to be safe. However, a medical practitioner may be exposed to low doses chronically as part of the work environment. Excessive cumulative x-ray radiation dosing is known to be harmful to a medical practitioner. The effects may be carcinogenic or genomic damage, which may be expressed many years from the time of exposure. Minimization of exposure to x-ray radiation is desirable and may be achieved following the principles of ALARA: “as Low as Reasonably Achievable.” Practices to comply with ALARA include maximized distance from the radiation source. A medical procedure, such as positioning of a bone or tissue access device, may require that the hand of a practitioner may be within or near the radiation field. Maximized distance from the radiation field may be achieved by providing an access device holder with an extended shaft to hold and orient the access device while the medical practitioner's hand is outside of the field.

Embodiments may be understood by reference to the drawings, wherein like parts are designated by like numerals throughout. It will be readily understood by one of ordinary skill in the art having the benefit of this disclosure that the components of the embodiments, as generally described and illustrated in the figures herein, could be arranged and designed in a wide variety of different configurations. Thus, the following more detailed description of various embodiments, as represented in the figures, is not intended to limit the scope of the disclosure, but is merely representative of various embodiments. While the various aspects of the embodiments are presented in drawings, the drawings are not necessarily drawn to scale unless specifically indicated.

It will be appreciated that various features are sometimes grouped together in a single embodiment, figure, or description thereof for the purpose of streamlining the disclosure. Many of these features may be used alone and/or in combination with one another.

The phrases “coupled to” and “in communication with” refer to any form of interaction between two or more entities, including mechanical, electrical, magnetic, electromagnetic, fluid, and thermal interaction. Two components may be coupled to or in communication with each other even though they are not in direct contact with each other. For example, two components may be coupled to or in communication with each other through an intermediate component.

The directional terms “distal” and “proximal” are given their ordinary meaning in the art. That is, the distal end of a medical device means the end of the device furthest from the practitioner during use. The proximal end refers to the opposite end, or the end nearest the practitioner during use. As specifically applied to the syringe portion of an inflation device, the proximal end of the syringe refers to the end nearest the handle and the distal end refers to the opposite end, the end nearest the inlet/outlet port of the syringe. Thus, if at one or more points in a procedure a physician changes the orientation of a syringe, as used herein, the term “proximal end” always refers to the handle end of the syringe (even if the distal end is temporarily closer to the physician).

“Tissue” is used in its broadest sense, to refer to any tissue or substance within the human body.

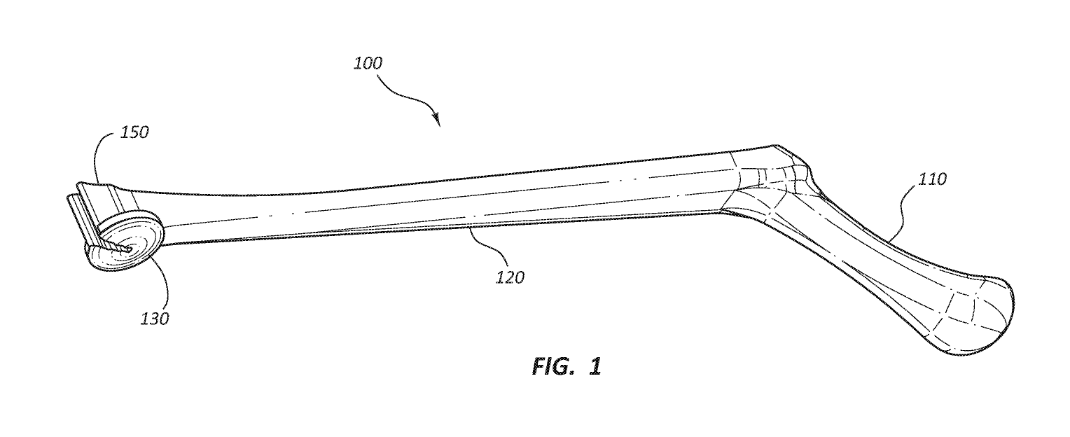

FIGS. 1-8 illustrate different views of access device holders and related components. In certain views each holder may be coupled to, or shown with, additional components not included in every view. Further, in some views only selected components are illustrated, to provide detail into the relationship of the components. Some components may be shown in multiple views, but not discussed in connection with every view. Disclosure provided in connection with any figure is relevant and applicable to disclosure provided in connection with any figure or embodiment.

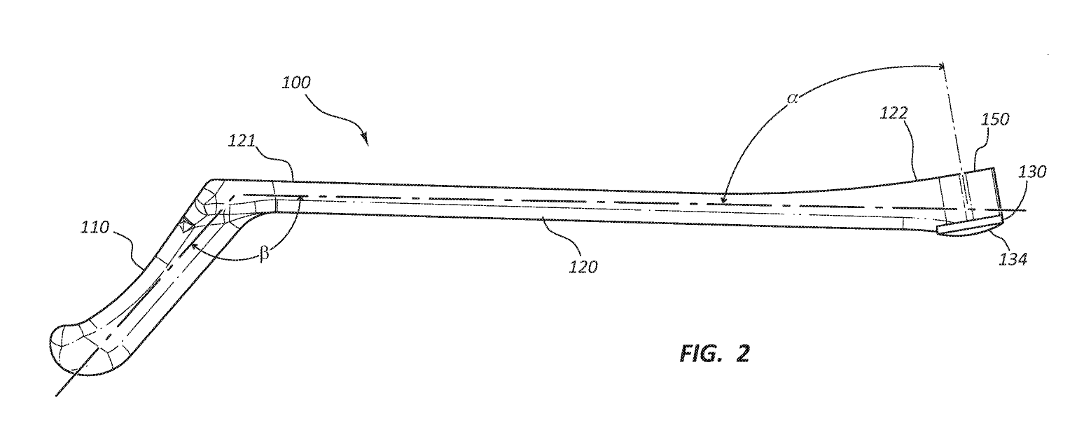

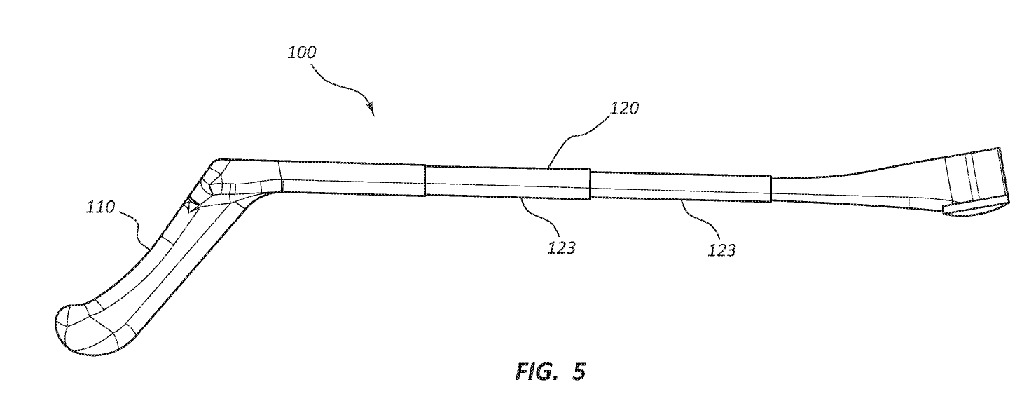

FIGS. 1-7C depict an embodiment of an access device holder 100. In the illustrated embodiment, the access device holder 100 is comprised of a handle 110, a shaft 120, a patient contact member 130, and a holding device 150. It is noted that certain subsets of these figures (e.g. FIG. 5) illustrate and describe a modified embodiment of the embodiment of FIGS. 1-2. For example, as detailed below, the embodiment of FIG. 5 comprises shaft 120 having an adjustable length, while the embodiment of FIG. 1-2 may not be adjustable. Nevertheless, for clarity, like elements are designated with like reference numerals throughout these embodiments. Referring to FIGS. 1-2, the handle 110 is disposed at the proximal end of the access device holder 100. The handle 110 is configured to be gripped by the hand of a medical practitioner. The handle 110 is coupled to the shaft 120 and may form a unitary unit with the shaft 120. The handle 110 may be coupled to the shaft 120 at a non-perpendicular angle β with the handle 110 extending proximally from the shaft 120. The coupling angle β, relative to horizontal plane of the shaft 120, may range from 105 degrees to 155 degrees including 120 degrees to 140 degrees. The handle 110 may comprise grip enhancing features to reduce slippage, such as bumps, ridges, recesses, roughened surface, etc. Additionally, the handle 110 may be partially formed from a soft or compliant material to enhance the gripping feature of the handle 110. The handle 110 may be formed from any suitable radiolucent material, such as a rigid or semi-rigid plastic material, and may be formed using known techniques such as injection molding, casting, machining, etc. The compliant material may be coupled to the handle 110 using known techniques, such as overmolding, spraying, dipping, etc.

The shaft 120 is configured to be coupled to the handle 110 to form a unitary unit and to extend distally from the handle 110. The shaft 120 may be wider at a proximal portion 121 than at a distal portion 122 and may be about the same thickness from the proximal portion 121 to the distal portion 122. The shaft 120 may be formed of any suitable radiolucent material, such as rigid or semi-rigid plastic material, and may be formed using known techniques, such as injection molding, casting, machining, etc. The shaft 120 may have a length ranging from 6 inches to 24 inches including 8 inches to 16 inches.

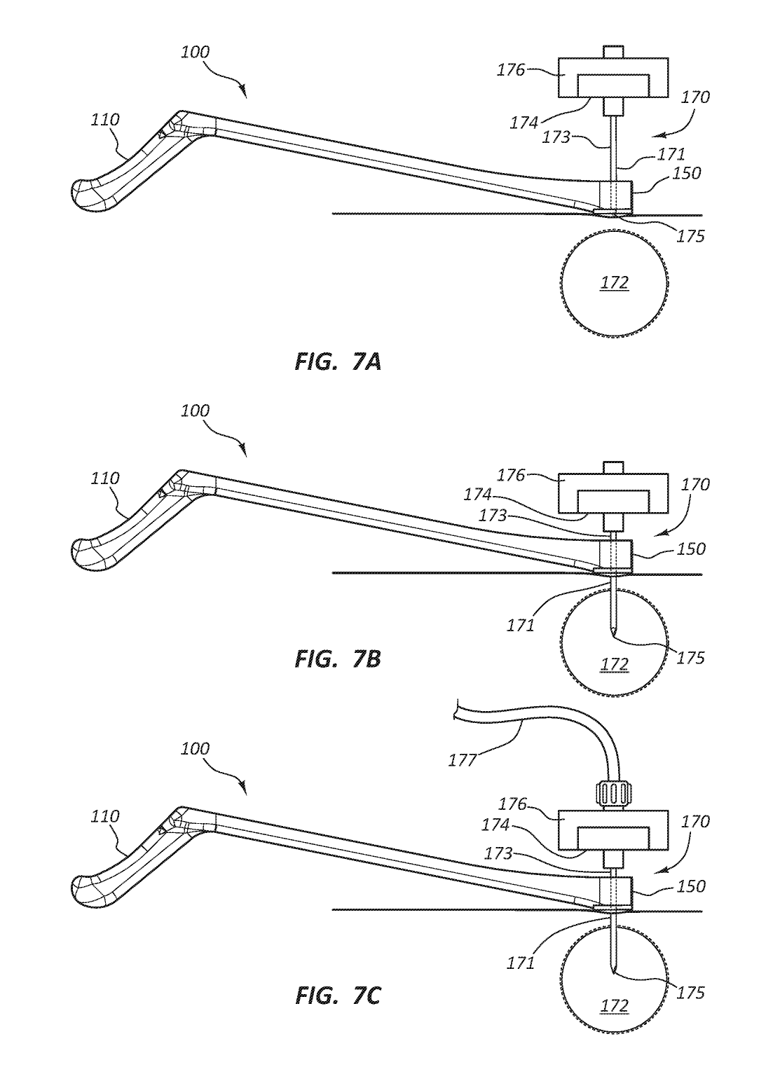

Referring to FIG. 5, in certain embodiments, the shaft 120 may be configured to have an adjustable length to accommodate a preferred working distance of the medical practitioner. The shaft 120 may comprise at least two telescoping segments 123. The segments 123 may be slidably disposed relative to one another and may be releasably locked together when in a retracted and an extended configuration. The segments 123 may be configured to be non-rotatable relative to the handle 110 and the holding device 150 to enable accurate orientation of an access device 170 (FIG. 7A). The shaft 120 may be adjustable to a length ranging from 6 inches to 24 inches.

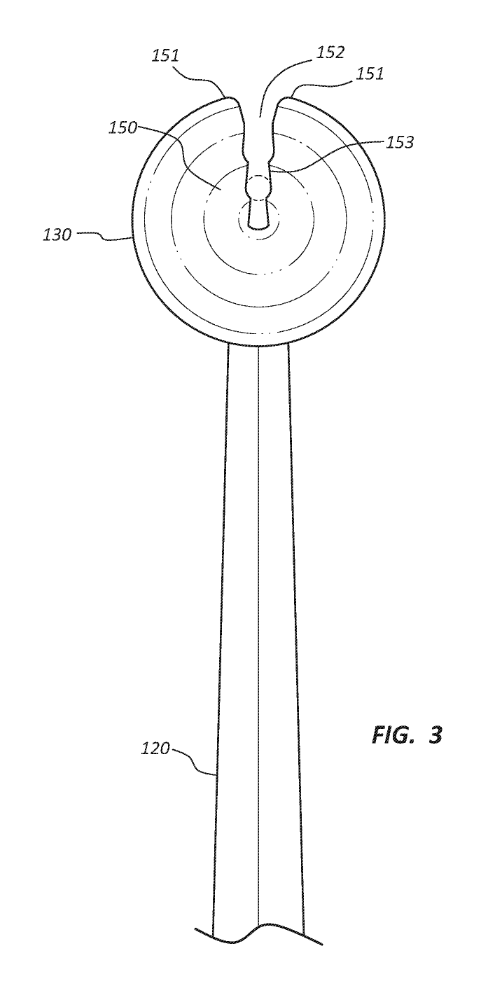

With continued reference to FIGS. 1-2, the holding device 150 may be configured to releasably hold the access device 170 at a predefined orientation relative to the handle 110. More specifically, the holding device 150 may be configured to engage with an elongate component or portion of the access device 170 such as the access device shaft 171 (FIGS. 7A-7C). The holding device 150 may be coupled to the distal portion 122 of the shaft 120 to form a unitary integrated unit. In certain embodiments, the holding device 150 may be releasably coupled to the distal portion 122 of the shaft 120 as will be discussed below. The holding device 150 may be deflected upward from the longitudinal axis of the shaft 120 so that in the defined orientation, the elongate component or portion of the access device of the access device 170 may be disposed at an angle α relative to the shaft 120. Referring to FIG. 3, in some embodiments, the holding device 150 may be configured with two prongs or engagement members 151 extending distally. A “V” shaped slot 152 may be configured between the prongs 151. The slot 152 may be configured with steps 153 such that the width of the slot 152 is incrementally smaller in the proximal direction. The smallest width of the slot 152 may be configured to be at the center of the longitudinal dimension of the holding device 150. Each incremental step 153 is configured to hold access devices 170 having different outer diameters. The slot 152 may hold access devices 170 ranging in diameter from 8 gauge to 17 gauge including 10 gauge to 13 gauge. The holding device 150 is placed over the bone or tissue to be accessed. Fluoroscopy, utilizing x-ray radiation, may be used to image the target bone or tissue. Therefore, the holding device 150 is radiolucent such that the target bone or tissue is not masked by the access device holder 100 and more specifically by the holding device 150. The holding device 150 may be formed from any suitable radiolucent material, such as rigid or semi-rigid plastic material, and may be formed using known techniques, such as injection molding, casting, machining, etc. The slot 152 may comprise a tacky or sticky material configured to enhance gripping of the access device 170 within the slot 152. The material may be a rubber or thermoplastic elastomer and may be coupled to the prongs 151 utilizing know techniques, such as overmolding, spraying, dipping, etc.

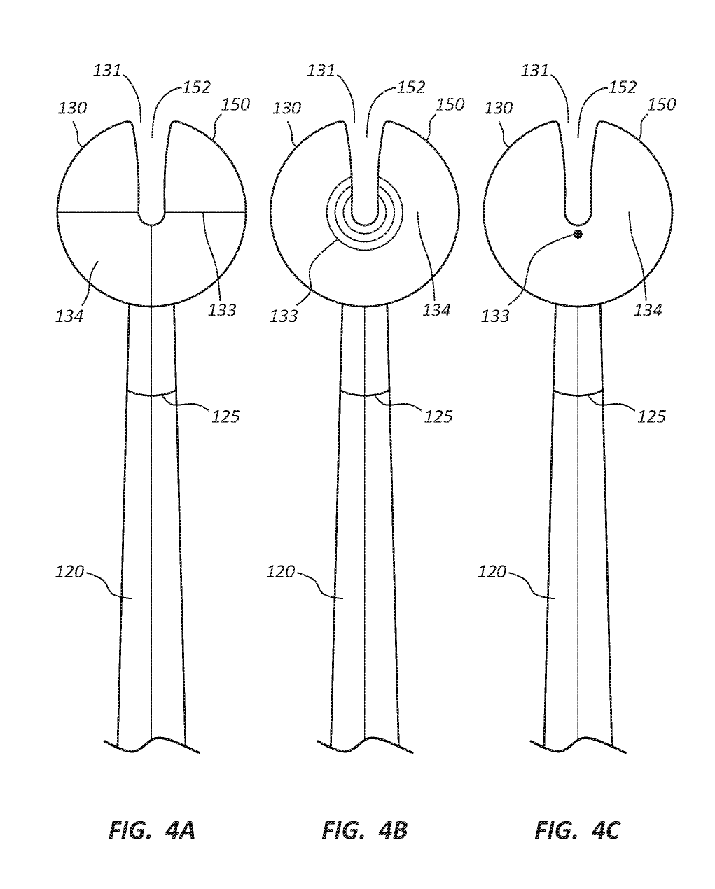

Referring now to FIGS. 4A-4C, in some embodiments, the holding device 150 may comprise a slot 152 configured to hold access devices 170 having a single outer diameter. In other words, multiple holding devices 150 configured with slots 152 of varying width may be provided to the medical practitioner. The holding device 150 and the distal portion 122 of the shaft 120 may be configured to be releasably coupled. In use, the medical practitioner may select an access device 170 having a shaft diameter preferred for the medical procedure. The medical practitioner may then select a holding device 150 configured with the slot 152 sized to hold the selected access device 170 and then couple the holding device 150 to the distal portion 122 of the shaft 120.

Referring to FIGS. 2-4C, in certain embodiments, the patient contact member 130 may comprise a convex surface 134. The patient contact member 130 may be coupled to the holding device 150 to form a unitary unit. The patient contact member 130 may comprise a slot 131 so that an access device 170 disposed in the slot 152 may extend through the patient contact member 130. The patient contact member 130 is configured to provide stability to the access device holder 100 such that when the access device 170 is properly oriented, the patient contact member 130 provides a wide base to maintain the orientation of the access device 170 while the access device 170 is inserted into the target bone or tissue. The patient contact member 130 may be formed from any suitable radiolucent material, such as rigid or semi-rigid plastic material, and may be formed using known techniques, such as injection molding, casting, machining, etc. The convex surface 134 of the patient contact member 130 may comprise a tacky or sticky material configured to minimize slipping of the patient contact member 130 on a patient's skin. The material may be a rubber or thermoplastic elastomer and may be coupled to the patient contact member 130 utilizing know techniques, such as overmolding, spraying, dipping, etc.

Referring to FIGS. 4A-4C, in some embodiments, the patient contact member 130 may comprise a targeting element 133. The targeting element 133 may be formed as cross-hairs (FIG. 4A), a bullseye comprising concentric circles (FIG. 4B), or a sphere (FIG. 4C). Other forms and shapes are possible and included within the scope of this disclosure. The targeting element 133 may be formed from any suitable radiopaque material, such as gold, titanium, lead, barium sulfate, bismuth trioxide, etc. The targeting element 133 may be embedded into or coupled to the surface of the patient contact member 130 utilizing known techniques, such as insert molding, welding, spraying, gluing, etc.

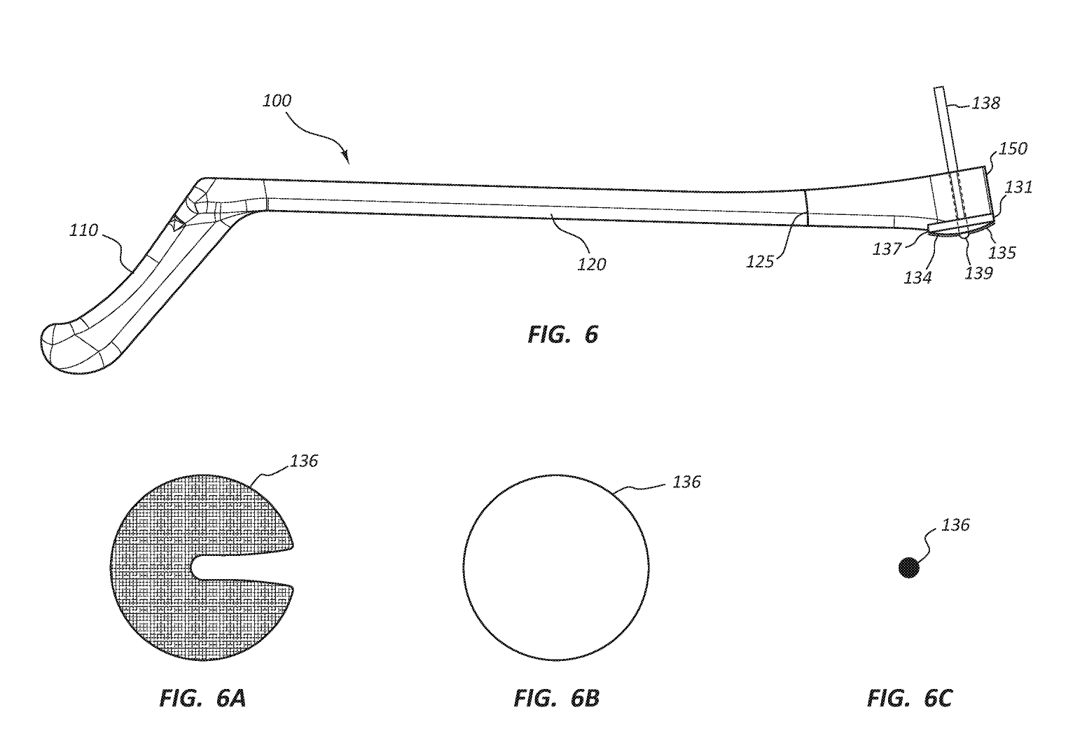

Referring to FIGS. 6-6C, in some embodiments, the patient contact member 130 and the holding device 150 may be configured to facilitate marking of a patient's skin to indicate the location the access device 170 may be inserted. The convex surface 134 of the patient contact member 130 may comprise an inked pad or stamp 135 configured to transfer an image 136 onto the skin of the patient at the location of the access device 170 insertion. The image 136 may be of the full convex surface 134 (FIG. 6A), or any other suitable image to indicate the insertion site of the access device 170. The ink used to create the image 136 may be a type of ink used for surgical site marking such that the ink is non-toxic to the patient and is not easily removed when the patient's skin is aseptically prepped. In other embodiments, an outer edge 137 of the patient contact member 130 may be traced utilizing a surgical marking pen 138 to provide the image 136 (FIG. 6B) for positioning the access device 170 over the insertion site. In yet another embodiment, the surgical marking pen 138 may be coupled to the holding device 150 such that a marking end 139 extends below the convex surface 134. The image 136 created by the surgical marking pen 138 may be a dot (FIG. 6C). The surgical marking pen 138 may be configured with different outer diameters to be held by the holding device 150.

In some embodiments, the patient contact member 130 is configured to facilitate orientation of the access device 170 held by the holding device 150 angular displacement about axes parallel and perpendicular to the shaft 120. The orientation directs the access device 170 to a target bone or tissue. Said another way, orientation aligns the longitudinal axis of the access device 170 with a predetermined tissue access path from the skin surface to the target tissue. A relative large movement of the handle 110 results in a relative small movement of the access device 170 because the access device 170 is positioned at or adjacent to the center point such that there is no lever arm or a very short lever arm. For example, the radius of the dome may range from 0.25 inches to 3.0 inches including 0.25 inches to 1.0 inches. The handle 110 may be disposed about 12 inches from the patient contact member 130. In this configuration, if the handle 110 is moved upward about 6 inches by the medical practitioner, the distal tip of access device 170 (FIG. 7A) moves proximally about 0.05 inches.

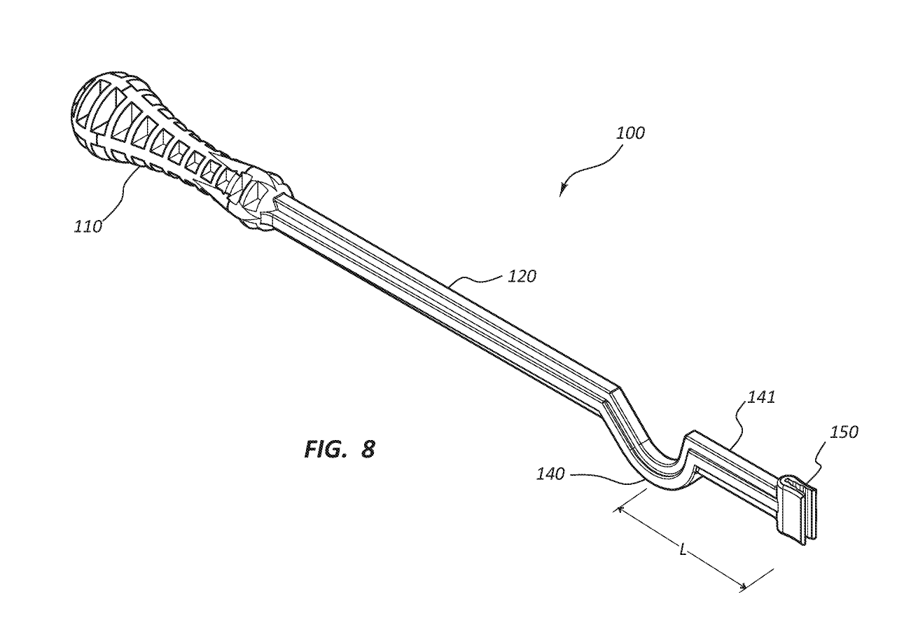

Referring to FIG. 8, a second embodiment of an access device holder 100 is illustrated. While like references numerals are used when referring to the embodiment of FIG. 8 as compared to the embodiment of FIGS. 1-2 (and other embodiments described above), it is noted that the access device holder 100 of the embodiment of FIG. 8 may comprise certain features not necessarily found in other embodiments and does not necessarily include all the elements of the prior described embodiments. Nevertheless, as with all embodiments, analogous disclosure may be equally applied between the various described embodiments. The access device holder 100 of FIG. 8 comprises a “U” shaped patient contact member 140 disposed proximal to the holding device 150. The patient contact member 140 is configured to rest on the skin of the patient such that as the handle 110 is manipulated by the medical practitioner, the holding device 150 and the access device 170 are displaced with less movement than the handle 110. The amount of movement is dependent upon the length “L” of a lever arm 141 from the patient contact member 140 to the access device 170.

FIGS. 7A-7C illustrate steps of a method of using the access device holder 100 to inject bone cement into a bone 172 in order to treat a fracture. The bone 172 may be a vertebral bone or any other type of bone where the injection of bone cement may be therapeutically beneficial. Alternatively, the access device holder 100 may be utilized to facilitate access to bone or other tissues for ablation or tissue biopsy. It is within the scope of this disclosure to include use of the access device holder 100 to facilitate access to any type of suitable human tissue for any type of suitable therapeutic or diagnostic purpose.

An exemplary method of using the access device holder 100 is as follows. The medical practitioner may obtain the access device 170 suitable to provide access to the vertebral bone 172 for the purpose of injecting bone cement into the vertebral bone 172 to provide stabilization of the fractured vertebral bone 172. The access device 170 may comprise a cannula 173, a cannula handle 174, a trocar 175 and a trocar handle 176. The trocar 175 is co-axially disposed within the cannula 173. The patient's skin over and surrounding the vertebral bone 172 is aseptically prepped and the area is draped with sterile drapes. The sterile access device holder 100 is obtained such that the holding device 150 is configured to securely hold the cannula 173 of the access device 170. The access device holder 100 may comprise either the holding device 150 configured to couple with the cannula 173 of different sizes or the holding device 150 configured to hold the cannula 173 of a single size such that the holding device 150 is interchangeable with other holding devices 150 configured to hold different size cannulas 173.

The medical practitioner grips the handle 110 and places the holding device 150 over the vertebral bone 172. A fluoroscopy imaging system is turned on and with the practitioner's hand outside of the x-ray radiation field, the holding device 150 is manipulated over the vertebral bone 172 using the fluoroscopy to provide a real time image of the targeting elements 133. When the targeting elements 133 (not shown) such as cross-hairs, concentric circles, sphere, etc., line up with a desired insertion location for the access device 170, the fluoroscopy is turned off and the access device holder 100 is held in place. The skin is marked with an image 136 (not shown) to indicate the insertion location. The skin may be marked by stamping the image 136, outlining the shape of the patient contact member 130 with the surgical marking pen 138, using the surgical marking pen held by the holding device 150, etc. The skin at the marked insertion site may be injected with a local anesthetic, such as lidocaine. A small incision at the marked insertion site may be made to facilitate insertion of the access device 170.

The access device 170 is coupled to the holding device 150 such that the tip of the trocar 175 does not extend below the bottom of the holding device 150. The medical practitioner grips the handle 110 and positions the holding device 150 and the access device 170 at the marked access site. The location of the holding device 150 is manipulated until it lines up with the image 136 on the patient's skin. The fluoroscopy is turned on and with the medical practitioner's hand outside of the x-ray radiation field, the fluoroscopy imaging is utilized to orient the access device 170 to be directed into the vertebral bone 172. The handle 110 is manipulated to orient the access device 170. When acceptable orientation of the access device 170 is achieved, the access device holder 100 is held in place with the patient contact member 130 providing stability to the access device holder 100 and the fluoroscopy is turned off. The access device 170 is inserted into the vertebral bone 172. A hammer may be used to impact the trocar handle 176 to drive the trocar 175 and cannula 173 into the vertebral bone 172. The access device holder 100 is decoupled from the access device 170. Proper placement of the trocar 175 and cannula 173 may be confirmed with fluoroscopy.

The trocar 175 is removed from the cannula 173. A bone cement delivery system is coupled to the cannula handle 174 which is in fluid communication with the cannula 173. The bone cement is delivered quickly through the cannula 173 into the vertebral bone 172 due to the short working life of the cement. The cannula 173 is removed from the patient.

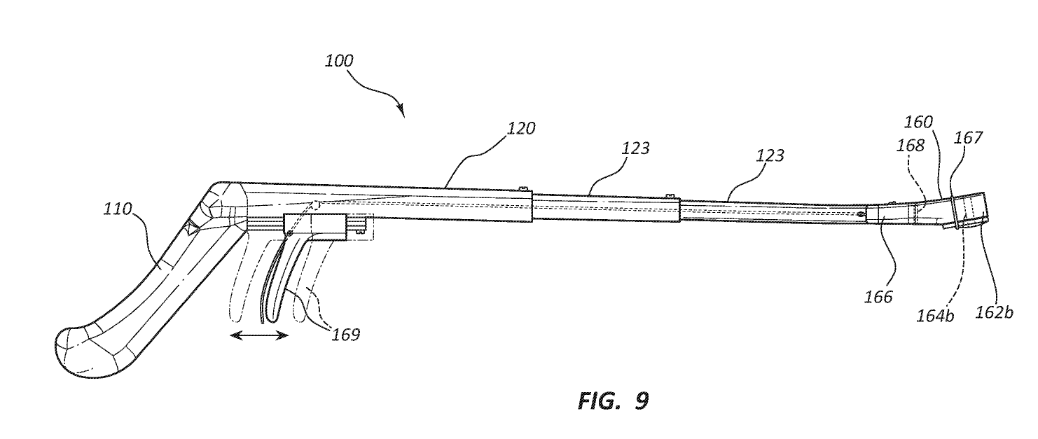

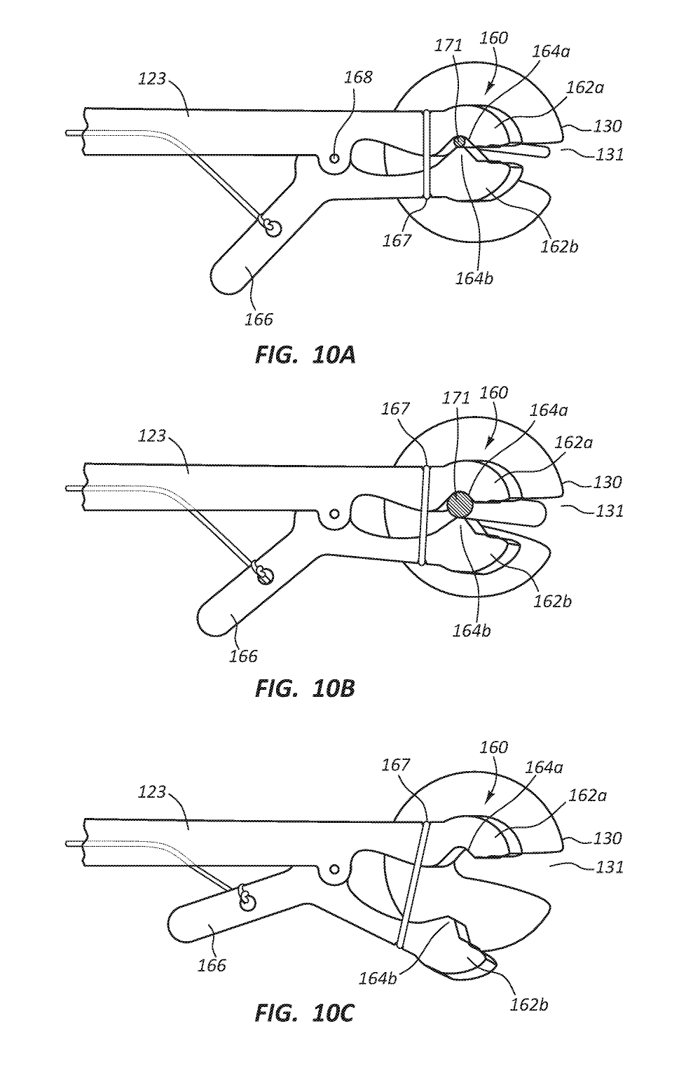

FIGS. 9-10C illustrate another embodiment of the access device holder 100 comprising features and components that in many respects may be similar to features and components of previous embodiments. While analogous disclosure is relevant to any embodiment shown herein, the embodiment of FIGS. 9-10C may not include every feature of prior embodiments and may comprise features not found in other embodiments. As shown, the embodiment of FIG. 9 may comprise a handle 110, a shaft 120, telescoping segments 123, a patient contact member 130, an actuatable holding device 160, and a depressible member 169. As further described below, the depressible member 169 may be operatively coupled to the holding device 160 via an actuation mechanism.

Referring to FIGS. 10A-C, the holding device 160 may be disposed in a secure configuration as shown in FIGS. 10A and 10B and a release configuration as shown in FIG. 10C. The secure configuration may comprise coupling of the access device holder 100 to the access device shaft 171 to control the orientation of the access device 170 by displacement of the handle 110. In other words, movement of the handle may directly result in orientation adjustment of the access device 170 and more specifically adjustment of one or more angles of the access device shaft 171 relative to a skin surface of a patient. The secure configuration may provide for orientation of the access device shaft 171 to align with the predetermined tissue access path and maintenance of the aligned orientation during at least a portion of the insertion process of the access device 170. The release configuration may comprise complete disengagement of the access device 170 so that the access device holder 100 may be physically separated from the access device 170. The holding device 160 may be detachably coupled to the shaft 120.

The access device holder 100 may be configured so that the holding device 160 can be manually transitioned between the secure configuration and the release configuration by the medical practitioner. As such, the access device holder 100 may comprise an actuation mechanism configured to functionally couple the depressible member 169 to the holding device 160. The depressible member 169 may be configured to be actuatable by the hand, thumb or fingers of the medical practitioner whereupon the holding device 160 is transitioned between the secure and release configurations. The depressible member 169 may also be partially actuated so as to partially transition the holding device 160 between the secure and release configurations. For example, in some instances, the medical practitioner may partially release the access device 170 to allow for sliding displacement of the access device 170 while maintaining orientation of the access device 170.

The actuation mechanism may comprise linkages, tension devices, rotating shafts, etc. to facilitate functional coupling between the depressible member 169 and the holding device 160. In some embodiments, the actuation mechanism may comprise a cable coupled to the depressible member 169 at a first end and an engagement member 162b at a second end such that movement of the depressible member 169 results in movement of the engagement member 162b. In other embodiments, the actuation mechanism may comprise a rotatable shaft disposed parallel to the shaft 120 such that actuation of the depressible member 169 rotates the rotatable shaft which results in rotation of one of the first and second engagement members 162a, 162b. Other components and methods of operation of the actuation mechanism may be defined by one of ordinary skill in the art having the benefit of this disclosure.

In some embodiments, the actuation mechanism may comprise components and/or features to enhance operation of the actuation mechanism such as detents, audible feedback, locks or latches, displacement stops, etc. For example, the actuation mechanism may comprise a lock disposed adjacent the depressible member 169 to lock the holding device 160 in one of the secure and release configurations. In other embodiments, the actuation mechanism may comprise one or more mechanical stops to prevent over displacement of the depressible member 169, one or both of the first and second engagement members 162a, 162b, or any other component. The actuation mechanism may be designed to accommodate length adjustment of the shaft 120. Any and all portions and/or components of the holding device 160 may be formed of radiolucent materials.

The access device holder 100 may be designed to allow the medical practitioner to transition the holding device 160 between the secure and release configurations from the proximal end 121, the distal end 122 or both. The access device holder 100 may comprise a depressible member 169 disposed at the proximal end 121 adjacent the handle 110 so that the medical practitioner may engage or disengage the access device 170 from a location outside the radiation field. The depressible member 169 may also facilitate engagement, disengagement and orientation of the access device 170 with a single hand.

The depressible member 169 may comprise any shape suitable for actuation by the medical practitioner such as a lever, knob, button, etc. The depressible member 169 may be configured to be pushed, pulled, pivoted and/or rotated by the thumb and/or one or more fingers of the medical practitioner. The depressible member 169 may be configured to facilitate simultaneous orientation of the access device 170 via the handle 110 and actuation of the depressible member 169 by the medical practitioner using a single hand.

The holding device 160 may comprise first and second engagement members 162A, 162B. The holding device 160 may be designed such that transitioning between the secure and release configurations comprises relative displacement of the first and second engagement members 162A, 162B or any portions thereof. The first and second engagement members 162A, 162B or any portions thereof may be in closer proximity to each other in the secure configuration than in the release configuration. As illustrated in FIG. 9, the engagement member 162B may be designed to pivot about the pivot point 168 to produce the relative displacement. However, the relative displacement of the first and second engagement members 162A, 162B may comprise translational displacement and/or rotational displacement about any axis of one or both of the first and second engagement members 162A, 162B. One or both of the first and second engagement members 162A, 162B may be functionally coupled to the depressible member 169. The access device holder 100 may also or alternatively comprise a depressible member 166 disposed at the distal end 122 providing the same functionality as depressible member 169.

The holding device 160 may comprise a biasing member 167 to provide a biasing force to at least one of the first and second engagement members 162A, 162B. The biasing force may be directed toward the secure configuration as illustrated in FIGS. 10A-C such that effort is required by the medical practitioner to dispose the holding device 160 toward the release configuration. The biasing member 167 may comprise a stretchable band, a coil spring, a leaf spring, a flexible portion of the holding device 160 or any other suitable component or mechanism for providing a force. In other embodiments, the biasing force may be directed toward the release configuration such that effort is required by the medical practitioner to dispose the holding device 160 toward the secure configuration.

The first and second engagement members may comprise complimentary engagement portions 164A, 164B. The engagement portions 164A, 164B may be configured to provide physical contact with the access device shaft 171 so as to secure the access device 170 in a predefined orientation relative to the handle 110 when the holding device 160 is disposed in the secure configuration. Either or both engagement portions 164A, 164B may comprise rigid components configured to make physical contact with the access device shaft 171 at discreet points. Alternatively, either or both engagement portions 164A, 164B may comprise flexible components and/or portions of components to facilitate defined lines of contact or defined areas of surface contact. The engagement portions 164A, 164B may also comprise compliant materials such as silicon, rubber, polyurethane, etc. The engagement portions 164A, 164B may be configured to couple to a range of diameters or perimeter configurations of various access devices or any other device having a rigid elongate portion as shown in FIGS. 10A-B.

The engagement portions 164A, 164B may be configured to establish a predefined friction force between the access device shaft 171 and the engagement members 162A, 162B when the holding device 160 is disposed in the secure configuration. The predefined friction force may or may not allow longitudinal displacement of the access device shaft 171 during the insertion process. In some instances, the holding device 160 may be disposed in contact with or in close proximity to the skin surface of the patient, in which instance the holding device 160 may facilitate longitudinal displacement of the access device shaft 171 relative to the holding device 160 during insertion. In other instances, the holding device 160 may be disposed above the skin so that during insertion the holding device 160 and access device shaft 171 move together. The friction may be great enough to at least partially insert the access device 170, e.g., through soft tissue, by forces manually exerted on the handle 110 by the medical practitioner from a location outside the radiation field. In some embodiments, the engagement portions 164A, 164B may also be configured to establish frictional engagement to facilitate rotational orientation of the access device 270 about a longitudinal axis of the access device shaft 171 to direct a curved distal portion of the shaft 171.

The patient contact member 130, as described above, may be configured to assist in orientation of the access device 170 and/or provide stability in maintaining the aligned orientation. The patient contact member 130 may detachably couple to the shaft 120 at the distal end 122 as shown in FIG. 9 or at a location disposed proximally from the distal end 122. The patient contact member 130 may also be configured to dispose the holding device 160 in close proximity to the skin of the patient or at a raised distance above the skin. In some instances the patient contact member may be detached and omitted from the access device holder 100 during use.

Use of the embodiment of FIGS. 9-10C, may comprise various steps in addition to the steps described above. The medical practitioner may couple the access device holder 100 to the access device 170, or other device such as a marking pen, by actuating the depressible member 169 to dispose the holding device 160 in the release state, inserting the access device 170 between the engagement portions 164a, 164b and releasing the depressible member 169 to allow the biasing member 167 to dispose the holding device 160 in the secure configuration. The medical practitioner may de-couple the access device holder 100 from the access device 170 by actuating the depressible member 169 to dispose the holding device 160 in the release configuration, separating the access device holder 100 from the access device 170 and releasing the depressible member 169 to allow the biasing member 167 to return the holding device 160 to the secure configuration. Alternatively, where an embodiment comprises a holding device 160 biased toward the release configuration, the medical practitioner may couple the access device holder 100 to the access device 170 by inserting the access device 170 between the engagement portions 164a, 164b, and actuating the depressible member 169 to dispose the holding device 160 in the secure configuration, and de-couple the access device holder 100 from the access device 170 by releasing the depressible member 169 to dispose the holding device 160 in the release configuration and separating the access device holder 100 from the access device 170.

The medical practitioner may at least partially insert the access device 170 into the patient while utilizing uninterrupted fluoroscopy. This may be performed by coupling the holding device 160 to a proximal portion of the access device shaft 171, placing the tip of the access device 170 at an insertion point within the radiation field, orienting the access device 170 to align with the predetermined tissue access path and applying forces to the handle 110 from a location outside the radiation field to create a downward force on the access device 170 sufficient to cause at least partial insertion.

The medical practitioner may adjust the coupling location on the access device shaft 171 of the access device 170 while the access device 170 is disposed within the radiation field. In some instances during the insertion process, the medical practitioner may find it desirable for the holding device 160 to be longitudinally displaced along the access device shaft 171. For example, if the holding device 160 is disposed on a distal portion of the access device shaft 171, the holding device 160 may be disposed in a partially released configuration to allow for longitudinal displacement as the access device 170 is inserted. Similarly, the medical practitioner may partially disengage the access device 170, move the holding device 160 proximally on the shaft, and reengage the access device 170 at a new location while the access device 170 is disposed within the radiation field.

Without further elaboration, it is believed that one skilled in the art may use the preceding description to utilize the present disclosure to its fullest extent. The examples and embodiments disclosed herein are to be construed as merely illustrative and exemplary and not a limitation of the scope of the present disclosure in any way. It will be apparent to those having skill in the art, and having the benefit of this disclosure, that changes may be made to the details of the above-described embodiments without departing from the underlying principles of the disclosure herein.

1. An instrument for holding a tissue access device, comprising:
an elongate shaft having a proximal end and a distal end and configured to dispose a tissue access device within a radiation field;
a holding device disposed at the distal end configured to couple with the tissue access device; and
a handle disposed at the proximal end,
wherein movement of the handle from a location outside of the radiation field orients the tissue access device disposed within the radiation field.

2. The instrument of claim 1, wherein the holding device comprises at least two engagement members configured to engage a range of perimeter configurations of elongate portions of tissue access devices.

3. The instrument of claim 2, wherein at least one engagement member is displaceable relative to the other engagement member between a secure configuration and a release configuration such that the holding device is coupled to the access device in the secure configuration and the holding device is decoupled from the access device in the release configuration.

4. The instrument of claim 3, wherein displacement of the at least one engagement member is manually actuatable from the proximal end.

5. The instrument of claim 4, wherein the at least one engagement member is biased toward at least one of the secure and release configurations.

6. The instrument of claim 1, further comprising a patient contact member coupled to the shaft.

7. The instrument of claim 6, wherein the patient contact member is detachably coupled to a portion of the shaft extending proximally from the distal end.

8. The instrument of claim 1, wherein the instrument comprises a radiopaque portion configured to visibly assist with alignment of the access device with a predetermined tissue access path.

9. The instrument of claim 1, wherein the holding device is detachably coupled to the shaft at the distal end.

10. The instrument of claim 1, wherein a length of the shaft is adjustable over a range.

11. The instrument of claim 1, wherein at least a portion of the instrument is formed from a radiolucent material.

12. A system for accessing tissue within a radiation field, comprising:
a tissue access device; and
a holder configured to dispose the tissue access device within a radiation field and orient the tissue access device from a location outside the radiation field,
wherein the holder comprises a shaft having a handle disposed at a proximal end and a holding device disposed at a distal end, and
wherein the holding device is configured to releasably couple with the tissue access device.

13. The system of claim 12, wherein the holding device is configured to couple to the tissue access device in a predefined orientation relative to the handle.

14. The system of claim 12, wherein the holding device is actuatable by a user.

15. The system of claim 14, further comprising a depressible member configured to actuate the holding device from a location outside the radiation field.

16. A method of accessing bodily tissue of a patient, comprising:
obtaining an access device holder configured to hold a tissue access device within a radiation field;
coupling the holder with a tissue access device;
disposing the tissue access device within a radiation field;
orienting the tissue access device to align with a predetermined tissue access path; and
inserting the tissue access device into a target tissue.

17. The method of claim 16, wherein coupling the holder with the tissue access device comprises actuating a depressible member coupled to the holder.

18. The method of claim 16, wherein the access device comprises at least one of a bone access device, a biopsy needle, a trocar, a cannula and a bone cement delivery system.

19. The method of claim 16, wherein inserting the tissue access device into a target tissue comprises longitudinally displacing the tissue access device relative to the access device holder.

20. The method of claim 16, further comprising de-coupling the holder from the tissue access device wherein the de-coupling comprises actuating a depressible member disposed outside the radiation field.
